(19) United States
(12) Patent Application Publication
(10) Pub. No.: US 2002/0173462 A1
(43) Pub. Date: Nov. 21, 2002

(54) Fibrinogen binding protein

(76) Inventors: Maria K. Boden Wastfelt, Stockholm (SE); Jan-Ingmar Flock, Bromma (SE)

Correspondence Address: BURNS DOANE SWECKER & MATHIS L L P, POST OFFICE BOX 1404, ALEXANDRIA, VA, 22313-1404, US

(21) Appl. No.: 09/938,497

(22) Filed: Aug. 27, 2001

Related U.S. Application Data
(63) Continuation-in-part of application No. 09/276,141, filed on Mar. 25, 1999, now Pat. No. 6,299,879.

(30) Foreign Application Priority Data
Sep. 20, 1993 (SE) PCT/SE93/00759
Sep. 13, 1993 (SE) 9302955-01
Sep. 21, 1992 (SE) 9202720-0

(51) Int. Cl. A61K 38/17; C07H 21/04; C12N 7/00; C12P 21/02; C12N 1/21; C12N 15/74

(52) U.S. Cl. 514/12; 536/23.7; 530/350; 435/69.1; 435/252.33; 435/320.1; 435/235.1

(57) ABSTRACT

The present invention relates to fibrinogen binding proteins, and agents comprising those proteins, for use in immunization, for therapeutics and for diagnostic purposes.

### CROSS-REFERENCE TO RELATED APPLICATIONS

This Application is a continuation-in-part of 09/276,141, filed Mar. 25, 1999, the contents of which are incorporated herein by reference.

### DESCRIPTION

1. Technical field

The present invention relates to fibrinogen binding proteins, and agents comprising those proteins, for use in immunization, for therapeutics and for diagnostic purposes.

2. Background of the invention

Staphylococcus aureus is a persistent pathogen that causes serious community-acquired and nosocomial infectious. The range of disease produced by S.aureus is broad, including endocarditis, osteomyelitis and septic shock. The appearance of methicillin-resistance among S.aureus strains has made it urgent to find other ways to combat this micro-organism.

The ability of S.aureus to establish a niche in the host is a crucial step of its pathogenesis. S.aureus produces a number of cell surface localized binding protein, fibronectin binding proteins (FnBPs), collagen binding protein, fibrinogen binding proteins (FgBP) vitronectin binding protein and elastin binding protein among others. A recent suggestion is to term these proteins receptins. Receptins are proposed to contribute to the success of colonization and persistence at various sites of the host. Binding of S.aureus to Fg is mainly due to the cell associated protein clumping factors (Clf A and B) (1) (2). Also three extracellular proteins with ability to bind to fibrinogen are produced by S.aureus; coagulase (3), Efb (extracellular Fg binding protein). (This protein was previously called Fib or the 19 kDa protein; both nomenclatures will be used here) (4) and Eap (extracellular adherence protein). (This protein was previously called the 60 kDa protein; both nomenclatures will be used here) (5).

The Eap protein causes agglutination of the bacteria, due to its ability to rebind to the surface of S.aureus and because of a strong tendency of Eap to form multimeric aggregates. Eap has a broad binding range for plasma Proteins. It can bind to at least seven plasma proteins, including fibronectin, fibrinogen and prothrombin. Eap can also bind to itself; in fact, Eap can be purified by running a Sepharose column to which Eap has been coupled. Exogenously added Eap enhanced significantly the adherence of S.aureus to fibroblasts and epithelial cells (5), due to its dual affinity for both plasma proteins on the cell surface and the bacteria itself. A putative target on the bacterial surface for Eap is a neutral phosphatase to which Eap has affinty.

If adherence of S. aureus to host components is the first step of infection, its ability to internalize and survive intracellularly, thereby escaping humoral immunity, becomes probably the second most important function for long term persistence. Several Gram positive bacteria, including among others Listeria monocytogenes, Streptococcus pyogenes and Enterococcus faecalis evade their host's immunity by internalizing into host cells. Listeria monocytogenes uses two invasion proteins for entry into mammalian cells, internalin A (InIA) and internalin B (InIB). InIA binds to E-cadherin, a transmembrane cell adhesion protein normally involved in homophilic cell-cell interactions. InIA promotes entry into the enterocyte-like epithelial cell line Caco-2. InIB interacts with the mammalian protein gC1q-R, the receptor of the globular part of the complement component C1q. Interestingly, InIB is not only cell associated but also found in culture supernatants of Listeria monocytogenes, analogous to Eap. It was also seen that InIB when added to the bacteria could rebind and enhance the internalization of Listeria monocytogenes into mammalian cells. Thus, the internalization process of Listeria monocytogenes is a multifactorial event. Similarly, at least three proteins involved in the internalization process are known from Streptococcus pyogenes, protein F1, M1 and M6. Enterococcus faecalis aggregation substance (AS) is expressed on the surface of the bacteria. It has been shown that AS aggregate bacteria and increase bacterial adherence and internalization to epithelial cells from the colon and duodenum but not from the ileum. Internalization of S. aureus into non professional phagocytic cells is well documented (6) (7) (8) (9). Less is known about the exact mechanism involved in the internalization process. Fibronectin Binding Protein (FnBP) was shown to be required for the internalization process into eukaryotic cells (10) (11) (12). It was proposed that FnBPs affinity for integrins covered with fibronectin would result in activation of host cell signal transduction pathways, which lead to actin-mediated phagocytosis of adherent bacteria (6) (10) (11). Although FnBPs obviously plays a crucial part in the internalization process, bacteria lacking FnBPs could still be internalized at a lower rate. Furthermore, no correlation was found between adherence ability and the amount of FnBPs produced by some S. aureus strains and Fn binding capacity only partly correlated with the ability of various strains of S. aureus to be internalized (10) (12). This indicates that the internalization process for S. aureus is complex and probably involves more than one factor.

Clumping of Staphylococcus aureus in plasma has been suggested as a potential virulence factor. Several mechanisms can be responsible for this phenomenon. A fibrinogen-binding protein has been suggested to cause aggregation of staphylococci in fibrinogen at the concentration found in plasma. The presence of protein A causes staphylococci to aggregate in normal human sera, which frequently contain specific immunoglobulins directed against staphylococcal antigens. Due to a high cell surface hydrophobicity, many staphylococcal strains autoaggregate under isotonic conditions. Clumping of staphylococci in fibrinogen is caused by clumping factor or fibrinogen-binding protein, situated on the staphylococcal cell surface (1). Fibrinogen has also been suggested to mediate adhesion of S. aureus to cultured human endothelial cells and to catheters in vitro and in vivo. Staphylococcal coagulase have been shown to induce polymerization of fibrinogen to fibrin by binding to prothrombin. The coagulase-prothrombin complex causes the release of fibrinopeptides from fibrinogen in a manner similar to that described for thrombin in physiological blood clotting.

We have described staphylococcal components that interact with fibrinogen and which can be purified from S. aureus culture supernatants (13) (14). These are a 87 kDa coagulase, a 19 kDa fibrinogen-binding protein, also termed Efb and a 60 kDa protein, also termed Eap. The 87, 19 and 60 kDa fibrinogen-binding proteins are essentially extracellular proteins, but can to some extent be found on the staphylococcal cell surface.

### BRIEF DESCRIPTION OF THE DRAWINGS

FIG. 1. Coomassie blue-stained SDS-PAGE of fibrinogen-binding material, affinity purified from S. aureus culture supernatants. Cells were grown in LB under low aeration conditions and samples were taken every hour. Lanes 1-6 represent samples taken after 1, 2, 3, 5, 7 and 9 h.

FIG. 2. Analysis of affinity-purified material from fibrinogen- and prothrombin-Sepharose. (a) Coomassie blue stained, undiluted eluate; (b) Immunoblot of eluate (diluted 1/100), probed with fibrinogen (10 μg/mi) and preabsorbed antifibrinogen antibody; (c) immunoblot of eluate (diluted 1/100), probed with prothrombin (10 μg/ml) and pre-absorbed antiprothrombin antibody. Lanes: 1, eluate from fibrinogen-Sepharose purified from culture supernatants of staphylococci grown in BHI for 3-4 h; 2, eluate from prothrombin-Sepharose purified from culture supernatants of staphylococci grown in LB for 6-8 h and initially passed through fibrinogen-Sepharose.

FIG. 3. Immunoblot analysis of eluate from fibrinogen-Sepharose. Lanes: 1, eluate (undiluted) incubated with fibrinogen (20 ng/ml) and antifibrinogen antibody; 2, eluate (undiluted) incubated with anti-19 serum.

FIG. 4. Immunoblot analysis of eluate (diluted 1/100) from fibrinogen- and prothrombin-Sepharose prepared as indicated in FIG. 2. (a) Anti-19 serum pre-absorbed with the 60-kDa protein- (b) Anti-19 serum pre-absorbed with the 19-kDa protein. Lanes: 1, eluate from fibrinogen-Sepharose; 2, eluate from prothrombin-Sepharose.

FIG. 5. Analysis of purified proteins eluted from preparative SDS-PAGE gels. (a) Silver stain of undigested sample; (b-d) Immunoblots probed with fibrinogen and antifibrinogen antibodies; (b) undigested sample; (c) samples digested with -chymotrypsin; (d) samples digested with staphylococcal V8 protease. Lanes: 1, 19 kDa protein; 2, 87 kDa protein; 3, 60 kDa protein.

FIG. 6. Analysis of affinity purified material from fibrinogen-Sepharose. Arrows indicate molecular masses (in kDa). Immunoblot probed with anti-19 serum. Lanes: 1, fibrinogen-proteins from S. aureus strain Newman; 2, fibrinogen-proteins from S. aureus strain FDA 486; 3, fibrinogen-proteins from E. coli XL-1 harboring plasmid pBfibill; 4, fibrinogen-proteins from E. coli XL-1 harboring plasmid pBfibT.

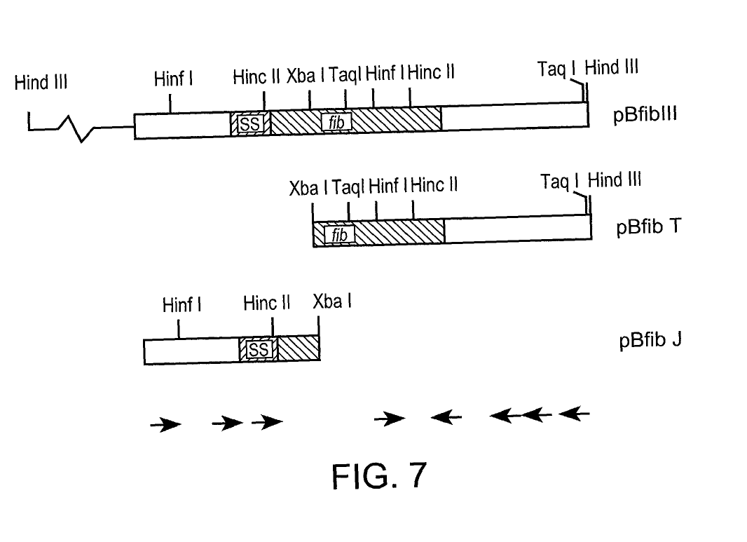

FIG. 7. Restriction map and sequencing strategy of the insert containing the efb gene. Subcloning of the efb gene from the original clone on a HindIII-HindIII fragment resulted in the pBfibill vector. This was further subcloned into the pBfibT and pbfib J vectors. Boxes show the regions for which the sequence was deduced. SS denotes the signal sequence and efb the structural gene for the mature Efb protein. Arrows indicate the primers used for sequencing.

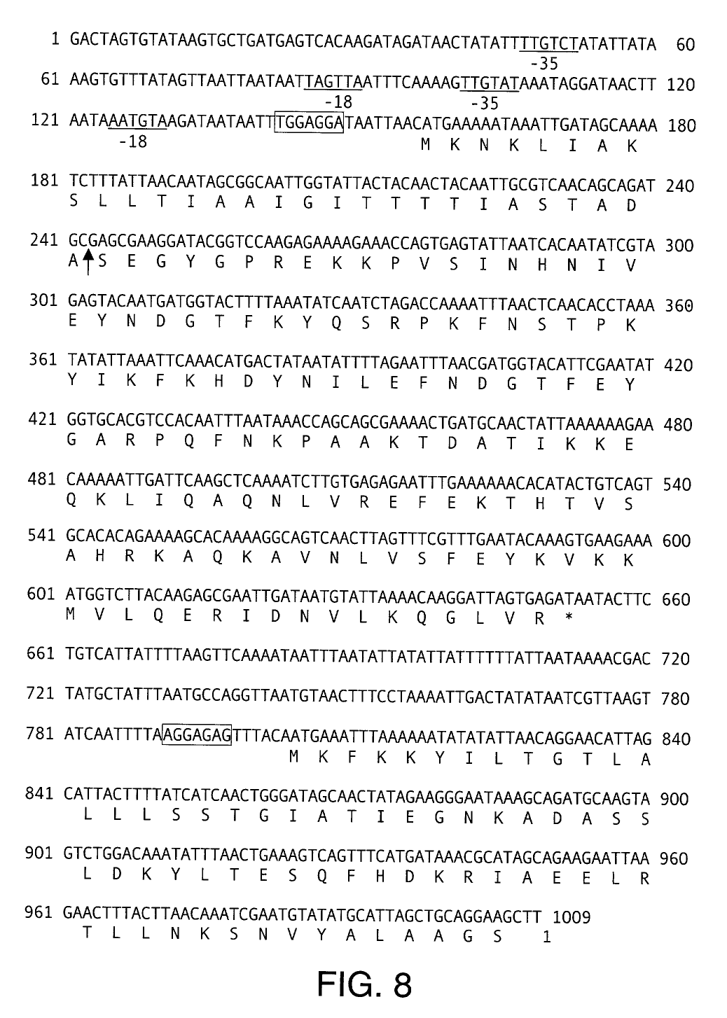

FIG. 8. Nucleotide and amino acid sequence for the fib protein gene. The box denotes a possible Shine-Dalgarno sequence. Putative promoter sequences are underlined. The vertical arrow indicates the cleavage site of the signal sequence.

FIG. 9. Comparison of the nucleotide sequences for the fib gene from strain FDA 486 (ton sequence) and strain Newman. Similarity is shown by blank spaces, differences in sequence is indicated by the diverging nucleotide of the Newman fib gene.

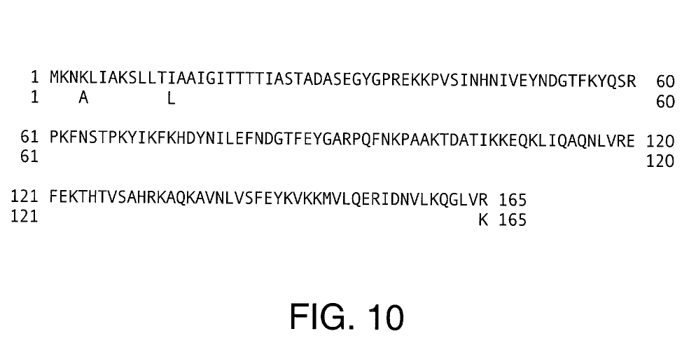

FIG. 10. Comparison of the amino acid sequences for the fib protein from strain FDA 486 (top sequence) and strain Newman. Similarity is shown by blank spaces, differences in sequence is indicated by the diverging amino acid of the Newman protein.

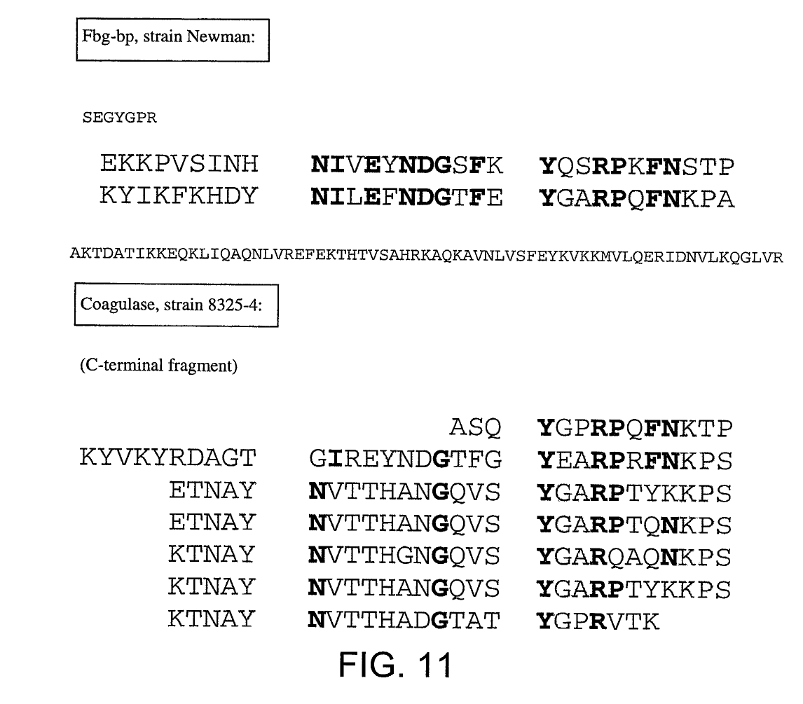

FIG. 11. sequence homology between the fib protein and the coagulase from S. aureus. Bold letters show homologies between the two repeats in the fib protein. Shaded letters show homologies between the fib protein and coagulase.

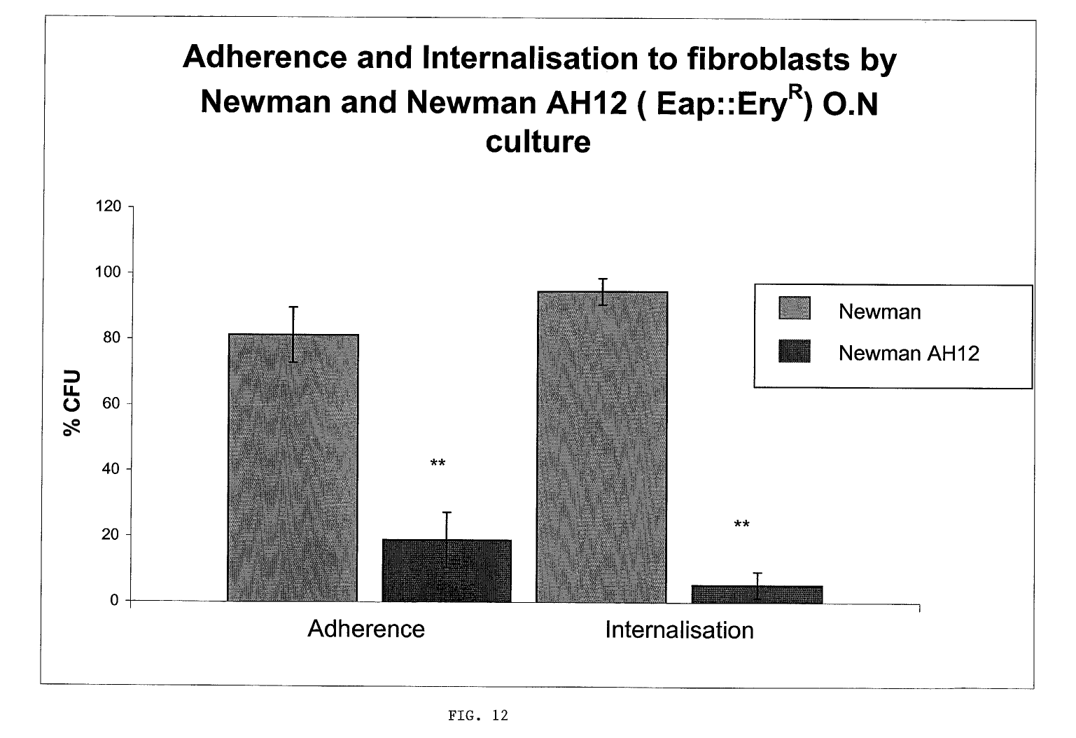

FIG. 12. Adherence to and internalization by fibroblasts of S. aureus Newman and Newman AH12 (Eap::Ery^{R}) O.N. culture.

FIG. 13. Adherence to and internalization by epithelial cells of S. aureus Newman and Newman AH12 (Eap::Ery^{R}), O.N. culture FIG. 14. Adherence to and internalization by epithelial cells of a 2 hours culture of S. aureus Newman and Newman AH12 (Eap::EryR).

FIG. 15. Internalization by fibroblasts of S. aureus strain Newman in the presence and absence of Eap-AB.

### DETAILED DESCRIPTION OF THE PREFERRED EMBODIMENTS OF THE INVENTION

The invention relates to the use of fibrinogen binding proteins in immunization, whereby the proteins, preferably in combination with a fusion protein in order to form a larger antigen to react upon, are injected in doses creating an immunological reaction in the host mammal. Thus the fibrinogen binding proteins can be used in vaccination of mammals to protect against infections caused by staphylococcal infections. Antibodies against fibrinogen binding proteins, such as Efb and Eap, can also be given to mammals as passive immuno prophylaxis or therapy.

Further, the fibrinogen binding proteins, or shorter peptides thereof, according to the invention can be used to block an infection in e.g. an open skin lesion. Wounds can be treated by using a suspension comprising the fibrinogen binding protein. Thus the fibrinogen binding proteins can be used to treat wounds, e.g., for blocking bacterial binding sites in fibrinogen, or for immunization (vaccination). In the latter case the host produces specific antibodies which can protect against attachment by bacterial strains comprising such fibrinogen binding proteins. Hereby the antibodies block the adherence of the bacterial strains to damaged tissue. Such antibodies directed against fibrinogen binding proteins could also inhibit other activities exerted by these staphylococcal proteins such as internalization of staphylococci into host cells.

Examples of colonizing of tissue damage are:

a) colonizing of wounds in skin and connective tissues, which wounds have been caused by a mechanical trauma, chemical damage, and/or thermal damage;

b) colonizing of wounds on mucous membranes such as in the mouth cavity, or in the mammary glands, urethra or vagina;

c) colonizing of connective tissue proteins, which have been exposed by minimal tissue damage (microlesions) in connection with epithelium and endothelium (e.g. mastitis, heart valve infection, hip exchange surgery).

When using the present fibrinogen binding proteins prepared by isolation from living cells, by means of hybrid-DNA technique, or synthesized, for immunization (vaccination) in mammals including humans, the proteins, or polypeptides thereof, are dispersed in sterile isotonic saline solution, optionally while adding a pharmaceutically acceptable dispersing agent. Different types of adjuvants can further be used in order to sustain the release in the tissue, and thus expose the protein for a longer period of time to the immune system of a body.

A suitable dose to obtain immunization is 0.5 to 5 μg of fibrinogen binding protein per kg body weight and injection at immunization. in order to obtain durable immunization, vaccinations should be carried out at consecutive occasions with an interval of 1 to 3 weeks, preferably at three occasions. Adjuvants are normally not added when repeating the immunization treatment.

When using the present fibrinogen binding proteins or polypeptides thereof for local topical administration the protein is dispersed in an isotonic saline solution to a concentration of 25 to 250 μg per ml. The wounds are then treated with such an amount only to obtain a complete wetting of the wound surface. For an average wound thus only a couple of milliliters of solution are used in this way. After treatment using the protein solution the wounds are suitably washed with isotonic saline solution or another suitable wound treatment solution.

Further the fibrinogen binding proteins, or synthesized polypeptide thereof can be used to diagnose bacterial infections caused by Staphylococcus aureus strains, whereby a fibrinogen binding protein of the present invention is immobilized on a solid carrier, such as small latex or Sepharose beads, whereupon sera containing antibodies are allowed to pass and react with the fibrinogen binding protein thus immobilized. The agglutination is then measured by known methods. Further the fibrinogen binding protein or polypeptide can be used in an ELISA test (Enzyme Linked Immuno Sorbent Assay). Hereby wells in a polystyrene microtiter plate are coated with the fibrinogen binding protein and incubated over night at 4° C. The plates are then thoroughly washed using PBS containing 0.05% Tween 20, and dried. Serial dilutions of the patient serum made in PBS-Tween, are added to the wells, and are incubated at 30° C. for 1.5 hrs. After rinsing antihuman IgG conjugated with an enzyme, or a horseradish peroxidase, or an alkaline phosphatase is added to the wells and further incubated at 30° C. for 1.5 hrs. During these incubations IgG from patient serum, and added antihuman IgG-enzyme conjugate, respectively, has been bound thereto. After rinsing, an enzyme substrate is added, p-nitrophosphate in case of an alkaline phosphatase, or orthophenylone diamine substrate (OPD) in case a peroxidase has been used, respectively. The wells of the plates are then rinsed using a citrate buffer containing 0.055% OPD, and 0.005% H₂O₂, and incubated at 30° C. for 10 min. The enzyme reaction is stopped by adding a 4N solution of H₂SO₄ to each well. The color development is measured using a spectrophotometer.

Depending on the type of enzyme substrate used a fluorescence measurement can be used as well.

Another method to diagnose S. aureus infections is by using the DNA sequence as the basis for a PCR diagnostic, a method well known in the art.

As used in the present application, the term “fibrinogen binding protein” includes any of polypeptide thereof as well, which constitute the minimal fibrinogen binding site of the complete protein.

The fibrinogen binding protein(s) can be used for raising antibodies by administering the protein and then isolating said antibodies, whereupon these are administered for passive immunization purposes.

### EXAMPLE 1

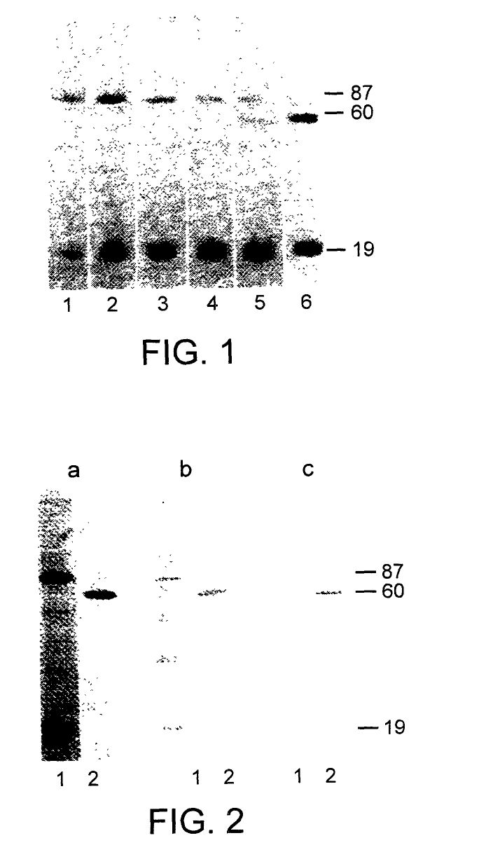

SDS-PAGE analysis of fibrinogen binding-proteins produced at different times during staphylococcal cell-growth. Staphylococcus aureus strain Newman was grown in BHI or LB and samples were taken every hour for 14 h. Culture supernatants were applied onto fibrinogen-Sepharose and the eluted material was analyzed on Coomassie blue-stained SDS-PAGE gels. FIG. 1 shows fibrinogen-binding proteins from culture supernatants of staphylococci grown in LB under low aeration conditions. Under these conditions, 87 kDa protein (coagulase) was produced in large amounts, mainly during the first 7 h and a 60 kDa protein (Eap) appeared after 5-6 h and was produced in large amounts after 9 h of growth. Under high aeration conditions, the 87 kDa protein was produced in lower amounts and the switch to production of the 60 kDa protein occurred after only 3 h, resulting in a higher production of 60 kDa protein compared to when less air was supplied to the culture. Using a rich medium like BHI, and the same high aeration conditions, this switch again occurred after 7 h (data not shown). In all cultures, the 87 kDa protein was produced mainly during the exponential growth phase and the 60 kDa protein mainly during the post-exponential growth phase. The switch from production of the 87 kDa protein to production of the 60 kDa protein reflected the nutritional status, rather than the optical density of the culture. A 19 kDa protein was produced constitutively during these 14 h of growth (FIG. 1).

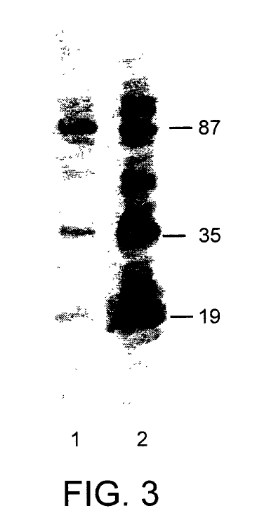

SDS-PAGE, affinity- and immuno-blot analysis of affinity purified proteins. Staphylococcus aureus grown In BHI for 3-4 h produced the 87 and 19 kDa proteins but no detectable 60 kDa protein. Such culture supernatants were applied onto fibrinogen-Sepharose in order to purify the 87 and 19 kDa proteins. Similarly, culture supernatants from S. aureus grown in LB for 6-8 h, containing predominantly the 60 kDa protein but also the 87 and 19 kDa proteins, were used to purify the 60 kDa protein. The crude material was first passed over fibrinogen-Sepharose, in order to eliminate the 87 and 19 kDa proteins, and the effluent (containing the 60 kDa protein which also bound to fibrinogen-Sepharose, but to a lower extent then the 87 and 19 kDa proteins) was applied onto prothrombin-Sepharose. The 87 and 19 kDa proteins did not bind to prothrombin-Sepharose. Eluted material from affinity purification was subjected to SDS-PAGE and affinity-blot analysis (FIG. 2). These blots were probed with fibrinogen or prothrombin, followed by rabbit antifibrinogen or rabbit antiprothrombin sera which had been pre-incubated with S. aureus culture supernatants in order to absorb naturally occurring antistaphylococcal antibodies. It could thus be shown that the 87 and 19 kDa proteins bound only to fibrinogen and not to prothrombin, while the 60 kDa protein bound both fibrinogen and prothrombin. Controls were performed by incubating filters with only preabsorbed primary antibody, omitting fibrinogen and prothrombin (data not shown). In these controls, no 87, 60 or 19 kDa proteins were detected. By using a dilution series both of antigen and fibrinogen or prothrombin, it was shown that the binding reactions were specific and not the result of contaminating blood proteins in the fibrinogen and prothrombin preparations. For example, 10 ng/ml of fibrinogen could detect 0.1 ng of the 87 or 60 kDa proteins in these affinity-blots. When 10 ng/ml of prothrombin was used in these tests, 0.1 ng 60-kDa protein could be detected, while a concentration of 10 μg/ml of prothrombin could not detect a 1 ng 87-kDa band (data not shown).

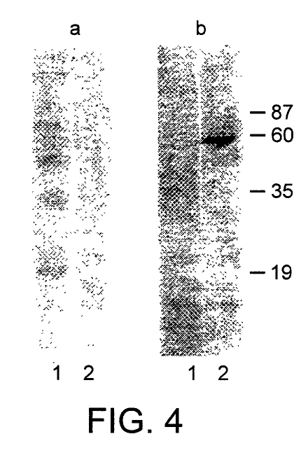

The anti-19 serum recognized not only the 19 kDa protein but also the 87 kDa protein and a 35 kDa protein (FIG. 3). Furthermore, there was a close resemblance between blots incubated with fibrinogen followed by anti-fibrinogen antibody and blots incubated with anti-19 serum.

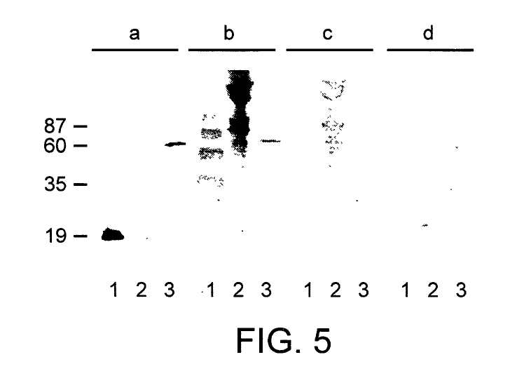

Antibodies to the 60 kDa protein seem to occur naturally among several mammalian species (e.g. rabbit, goat and man; data not shown). The anti-19 serum, as well as pre-immune serum from the same rabbit, showed some reactivity towards this 60 kDa protein. However, pre-absorption with 19 kDa protein completely abolished binding to the 19 and 35 kDa bands, but not to the 60 kDa band, while antiserum pre-absorbed with 60 kDa protein reacted with the 19 and 35 kDa bands but not with the 60 kDa band (FIG. 4).

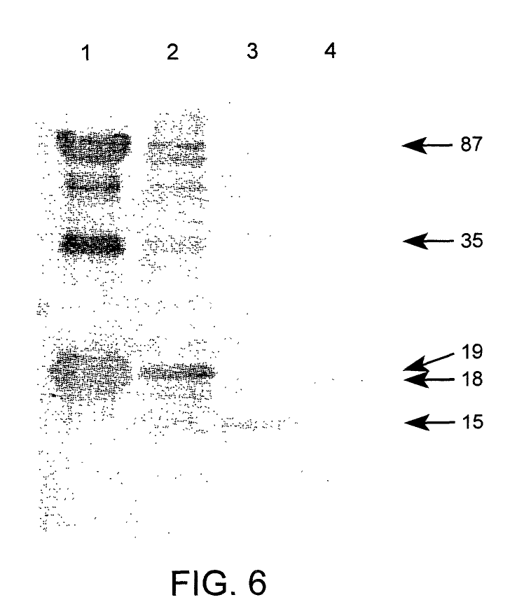

Peptide mapping. Proteins were purified by a combination of affinity chromatography and preparative SDS-PAGE. The purity of these preparations was confirmed on silver stained SDS-PAGE gels (FIG. 5). Dimerization of the 19 kDa protein into a 35 kDa protein could be detected on the silver stained gels. On affinity-blots, using fibrinogen and antifibrinogen antibodies, not only the 35 kDa dimer, but also bands of higher molecular weight were detected. Upon digestion with -chymotrypsin, the dimerization of the 19 kDa protein was disrupted, but the 19 kDa band was left intact. This protease did not have any apparent effect on the 87 kDa protein, whereas the fibrinogen-binding ability of the 60 kDa protein was completely lost after treatment with alfa-chymotrypsin. On the contrary, treatment of these proteins with staphylococcal V8 protease only partly digested the 60 kDa protein while the 87-kDa protein was digested into low molecular weight peptides (FIG. 5).

Discussion. We have previously described a 87 kDa fibrinogen-binding protein which exerts coagulate activity and is produced by S. aureus in culture supernatants. The 87 kDa coagulase was produced early during growth and was later replaced by the 60 kDa protein. The rate at which this switch occurred varied with growth rate and type of media used, i.e. under low aeration conditions or in a rich medium this switch was postponed (data not shown). This suggests that the presence of some environmental factor(s) induces the production of the 87 kDa protein and suppresses 60 kDa protein production.

It was concluded from the results of the analyses by SDS-PAGE and immunoblotting of proteins purified by affinity chromatography that both the 60 and 87 kDa proteins bound fibrinogen, but only the 60 kDa protein bound prothrombin (FIG. 2). We have shown that contamination with 1 ng/ml fibrinogen can detect band of 100 ng of fibrinogen-binding protein in immunoblot experiments. When antigens were diluted to 1 or 0.1 ng per band and ligands were used at 10 ng/ml, background due to contamination in these preparations was eliminated (data not shown).

Thus the following nucleotide sequence is present in the gene coding for the Efb protein:

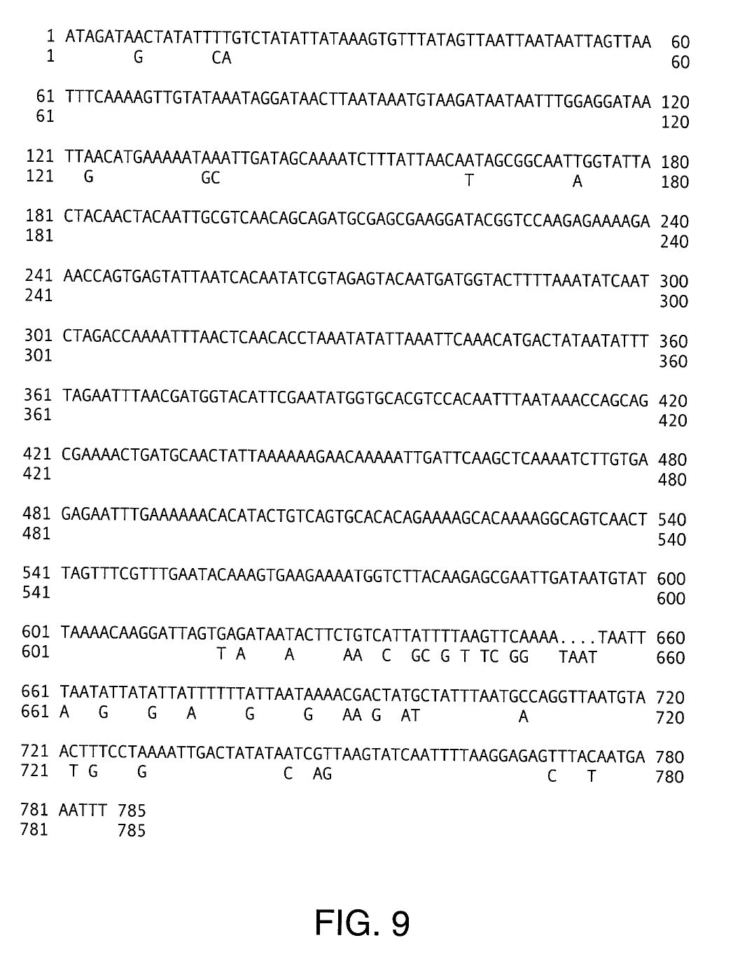

GAGCGAAGGA TACGGTCCAA GAGAAAAGAA ACCAGTGAGT
ATTAATCACA ATATCGTAGA GTACAATGAT GGTACTTTTA
AATATCAATC TAGACCAAAA TTTAACTCAA CACCTAAATA
TATTAAATTC AAACATGACT ATAATATTTT AGAATTTAAC
GATGGTACAT TCGAATATGG TGGAGGTCCA CAATTTAATA
AACCAGCAGC GAAAACTGAT GCAACTATTA AAAAAGAACA
AAAATTGATT CAAGCTCAAA ATCTTGTGAG AGAATTTGAA
AAAACACATA CTGTCAGTGC ACACAGAAAA GCACAAAAGG
CAGTCAACTT AGTTTGGTTT GAATACAAAG TGAACAAAAT
GGTCTTACAA GAGCGAATTG ATAATGTATT AAAACAAGGA
TTAGTGAGA

whereby this nucleotide sequence encodes for the following protein starting at nucleotide 243: (In FIG. 8 nucleotides 156-242 encode a signal peptide.)

SEGYGPREKK PVSINHNIVE YNDGTFKYQS RPKFNSTPKY
IKFKHDYNIL EFNDGTFEYG ARPQFNKPAA KTDATIKKEQ
KLIQAQNLVR EFEKTHTVSA HRKAQKAVNL VSFEYKVKKM
VLQERIDNVL KQGLVR

Although antisera to the 19 kDa protein recognized the 87 kDa protein (FIG. 3), pre-absorption with 19 kDa protein which could eliminate the binding to the 19 kDa protein, could not completely abolish this binding to the 87 kDa protein. In addition, antisera to the 87 kDa protein did not specifically recognize the 19 kDa protein (data not shown). The immunological cross-reactivity could be due to structural similarities in the fibrinogen-binding sites of these proteins. Antisera to the 19 kDa protein also recognized the 35 kDa protein (FIG. 3). We have previously shown that the 19 kDa protein spontaneously forms 35 kDa dimers (not reducible with 2-mercaptoethanol) and to a lesser extent higher molecular weight bands that seem to be trimers and tetramers of this protein (13). Minor bands in the preparation could thus be due to further aggregation of the 19 kDa protein or to degradation of the 87 kDa protein. By pre-absorbing the rabbit anti-19 serum with either 19 or 60 kDa proteins, it was shown that there were no shared antigenic epitopes between the 60 kDa protein (Eap) and 19 kDa protein (Efb) (FIG. 4). It is likely that antibodies against the 60 kDa protein are present in most normal rabbit sera. This reactivity is not due to unspecific binding to immunoglobulins. The purified 60 kDa protein did not bind control antibodies in immunoblots, and was thus shown not to contain protein A activity.

Peptide mapping analysis suggested that the 87, 60 and 19 kDa proteins are not closely related (FIG. 5). It was shown that digestion with alfa-chymotrypsin and staphylococcal V8 protease gave different peptide banding patterns with the three different proteins, and that the 60 kDa protein completely lost its ability to bind fibrinogen upon digestion with alfa-chymotrypsin, whereas the 87 and 19 kDa proteins were unaffected.

In conclusion, S. aureus strain Newman produces three distinct fibrinogen-binding proteins, one of which is coagulase. These are produced in a sequential manner during growth and have different binding and antigenic properties. The fibrinogen-binding protein of 19 kDa can spontaneously forms dimers and larger aggregates. The role of fibrinogen-binding proteins in staphylococcal virulence and pathogenicity has not yet been established. However, in our preliminary study, 90% of 40 S. aureus isolates from wound infections have coagulase activity, and among these >60% produced the 87 kDa protein. It is notable that fibrinogen binding proteins are produced in large amounts by S. aureus and in such a fashion that there is always one type of fibrinogen binding protein present in the culture medium.

### EXAMPLE 2

Binding of staphylococci to fibrinogen on coated coverslips or on catheters has been described. It is also a well known fact that most Staphylococcus aureus clump in the presence of fibrinogen. It has been suggested that this clumping reaction involves a small peptide at the COOH-terminal part of the gamma chain on the fibrinogen molecule. We have identified 3 different fibrinogen-binding proteins from Staphylococcus aureus, all of which can be found on the staphylococcal cell surface (14). However, these proteins cannot be described as cell surface proteins because they are mainly expressed extracellularly. In addition one of the identified fibrinogen-binding proteins was found to be coagulase, a well known extracellular staphylococcal protein. The other fibrinogen-binding proteins were of 60 kDa and a 19 kDa fibrinogen-binding protein without coagulase activity.

### Materials and methods

Bacterial strains and culture conditions. Staphylococcus aureus Newman was kindly provided by M. Lindberg, Swedish University of Agricultural Sciences, Uppsala, Sweden. Staphylococci were grown overnight in Brain Heart infusion (BHI) medium (Difco Laboratories, Detroit, Mich.) or in Luria-Bertani (LB) medium. After centrifugation, the bacterial pellet was resuspended in 20 culture volumes of freshly prepared BHI or LB and grown at 37° C. with constant shaking in Ehrlenmeyer flasks (low aeration) or in dented flasks (high aeration).

Affinity chromatography. Staphylococcal proteins are affinity purified as described previously.¹⁰ Briefly, fibrinogen-Sepharose and prothrombin-Sepharose were prepared by coupling human fibrinogen (IMCO, Stockholm, Sweden) or human prothrombin (Sigma Chemical Co, St. Louis, Mo.) to CNBr-activated Sepharose 4B (Pharmacia Uppsala, Sweden), by the procedure recommended by the manufacturer. The Sepharose was equilibrated with phosphate-buffered saline (PBS;145 mM NaCl, 10 nM phosphate, ph 7.4) containing 0.05% Nonidet P-40. Staphylococcal culture supernatants supplemented with 1 mM phenylmethylsulfonyl fluoride (PMSF), 10 mM EDTA and 0.05% Nonidet P-40 were applied. The absorbed material was eluted with 0.7% acetic acid containing 0.05% Nonidet P-40. The eluted material (eluate) was concentrated in Centricon microconcentrators (Amicon, Danvers, Mass.) or by acetone precipitation.

SDS-PAGE, affinity- and immuno-blotting. SDS-PAGE and subsequent diffusion blotting was performed using the PhastSystem (Pharmacia) as described previously.¹⁰ Nitrocellulose filters were incubated for 1 h at room temperature with human fibrinogen or human prothrombin at concentrations between 1 ng/ml and 10 μg/mi in PBS supplemented with 0.05% Tween 20. Primary antibodies rabbit anti-human fibrinogen (Dakopatts, Glostrup, Denmark), rabbit anti human prothrombin (Dakopatts), and rabbit anti-19 kDa protein were diluted 1:1000 and incubated with the filters for 2 h. The rabbit anti-19 kDa protein antibodies (anti-19 serum) were obtained by subcutaneous immunization of rabbits with a highly purified 19 kDa protein preparation emulsified in complete Freund's adjuvant. In order to eliminate naturally occurring antistaphylococcal antibodies in rabbit antifibrinogen or rabbit antiprothrombin antisera, these were pre-absorbed with staphylococcal culture supernatants from cells grown in LB for 6 h. Undiluted antisera was added to 10 volumes of culture supernatant and incubated at room temperature for 1 h or at 4° C. for 4 h before diluting the antibody to the appropriate concentration. The anti-19 serum was absorbed with 19 or 60 kDa proteins purified from preparative gels. The gel slices were homogenized in PBS containing 0.1% Nonidet P-40 before being added in a 10-fold excess to the antisera and incubated as described above. Alkaline phosphatase (ALP) conjugated goat anti-rabbit immunoglobulin G antibodies (Sigma) were diluted 1:1000 and incubated with the filters for 1 h. The ALP reaction was developed in 100 n-14 Tris hydrochloride (pH 8.0) containing 10 nM MgCl₂, 0.02 mg -naphtylphosphate per ml (E. Merck AG, Darmstadt, Germany) and 0.02 mg Fast Blue (Merck) per ml for 10-20 min.

Purification of proteins. The 87, 60 and 19 kDa protein were purified from preparative SDS-PAGE gels by eluting proteins from gel slices in a Model 422 ElectroEluter (Bio-Rad, Hercules, Calif.).

Fragmentation of proteins by proteases. Proteins were digested with 40 μg/ml of alfa-chymotrypsin or staphylococcal V8 protease (Sigma) for 1 h on ice.

Determination of NH₂-terminal sequences. Samples were analyzed in a 470 Protein Sequencer (Applied Bioystems, Faster City, Calif.). Media and chemicals. E. coli were grown in Luria Bertani medium at 37° C. Ampicillin (50 μg/ml) and tetracycline (5 μg/ml) were added when appropriate. Restriction enzymes were purchased from Promega. IPTG and X-gal were from Boehringer-Mannheim. All other chemicals were purchased from Sigma (Sigma Chemical Co, St. Louis, Mo.) or Merck (E. Merck AG, Darmstadt, Germany).

Affinity chromatography. Staphylococcal proteins were affinity purified as described previously (Boden and Flock, 1989). Briefly, fibrinogen-Sepharose was prepared by coupling human fibrinogen (IMCO, Stockholm, Sweden) to CNBr-activated Sepharose 4B (Pharmacia, Uppsala, Sweden), by the procedure recommended by the manufacturer. The Sepharose was equilibrated with phosphate-buffered saline (PBS; 145 nM NaCl, 10 mM phosphate, pH 7.4) containing 0.05% Nonidet P-40. Staphylococcal-culture supernatants supplemented with 1 mM phenylmethylsulfonyl fluoride (PMSF), 10 mM EDTA and 0.05% Nonidet P-40 were applied. The absorbed material was eluted with 0.7% acetic acid containing 0.05% Nonidet P-40. The eluted material was concentrated by acetone precipitation.

Sodium dodecyl sulphate polyacrylamide gel electrophoresis (SDS-PAGE), affinity- and immuno-blotting. SDS-PAGE and subsequent diffusion blotting was performed using the Phast System (Pharmacia) as described (13). Nitrocellulose filters are incubated for 1 hour at room temperature with human fibrinogen at 10 μg/ml in PBS supplemented with 0.05% Tween 20. Primary antibodies (rabbit antihumanfibrinogen (Dakopatts, Glostrup, Denmark) and rabbit anti-fib protein) were diluted 1:500 or 1:1000 and incubated with the filters or 2 hours. The rabbit anti-fib protein antibodies (antifib serum) were obtained by subcutaneous immunization of rabbits with a highly purified 19-kDa protein preparation emulsified in complete Freund's adjuvant. Alkaline phosphatase (ALV) conjugated goat anti-rabbit immunoglobulin G antibodies (Sigma) were diluted 1:1000 and incubated with the filters for 1 hour. The ALP reaction was developed in 100 Tris hydrochloride (pH 8.0) containing 10 mM MgCl₂, 0.02 mg -naphtylphosphate per ml (E. Merck AG, Darmstadt, Germany) and 0.02 mg Fast Blue (Merck) per ml for 10-20 min.

Incidence of FgBPs. The incidence of the 19 and the 87 kD FBPs were measured. Thirty-nine S. aureus isolates of human origin and thirty-seven bovine mastitis isolates, taken from a wide variety of sources, were tested by PCR for the gene and in affinity blotting for the proteins.

All (100%) of the human isolates were positive in both PCR and affinity blotting for the 19 kD protein and 95% were positive for the 60 kD tested by the same methods.

Vaccination. The 19 and 87 kD proteins in combination were used to immunize mice which were subsequently subjected to experimental mastitis caused by S. aureus. A control group was given only the adjuvant (Freund's). Histopathological examination and bacterial count was performed after 24 hours. A significant (p<0.05) difference in the number of colonizing bacteria was found between the two groups.

### EXAMPLE 3

The purpose of this study was to investigate the potential role of Eap in adherence and internalization of S. aureus. A mutant for the eap gene in S. aureus strain Newman (Newman AH12) was used and found to have significantly reduced ability to adhere to and internalize fibroblasts and epithelial cells as compared to the isogenic parental strain. Furthermore, Eap-antibodies were able to reduce the internalization of the native strain. The data provide evidence for an internalization pathway that involves the Eap protein of S. aureus.

### Materials and Methods

Bacterial Strains and Culture Conditions: S. aureus strain Newman, and S. aureus strain Newman AH12 (eap::Ery^{R}) were grown in Luria-Broth (LB) for two hours or overnight (ON) at 37° C. with shaking. The cells were washed with phosphate-buffer saline (PBS) and resuspended in PBS. Strains L12, L40, L167 (isolated from cases of endocarditis), U35, U61 and U98 (nasal colonizers) were cultured in the same way.

Purification of Eap. One liter of S. aureus strain Newman was grown O.N at 37° C. in LB medium. The cultures were centrifuged and FgBPs from the supernatant were isolated by affinity chromatography on Fg-Sepharose (Pharmacia, Uppsala) as described by Bodén and Flock (13) (14). Proteins were eluted with 0.7% acetic acid, dialyzed against 40 mM phosphate buffer, pH 6.5 (buffer A) and subjected to FPLC on a Mono S column (Pharmacia), using a gradient of 0 to 100% buffer B (1M NaCl in buffer A). Three peaks of proteins were eluted from the strain Newman. The first one eluted at a salt concentration of 0.15 to 0.25 M NaCl (coagulase), the second at 0.35 to 0.45 M NaCl (Efb) and the third peak at a concentration of 0.5 to 0.7 M NaCl (Eap). The eluate (third peak) was dialyzed in PBS

Binding and internalization of S. aureus strain Newman and Newman AH12 to fibroblasts and epithelial cells. Fibroblasts cells were cultured in Eagles medium (Gibco BRL) supplemented with 10% foetal calf serum (HyClone), Hepes Buffer, -glutamine, penicillin (100 U/ml) and streptomycin (100 U/ml). Epithelial cells (HACAT keratinocytes) were cultured in Dulbecco's Mod Eagel Medium (with sodium pyruvate, glucose and pyridoxine) supplemented with 10% fetal calf serum (HyClone), penicillin (100 U/ml) and streptomycin (100 U/ml). Cells (Fibroblasts and epithelial cells) were seeded (7.8×10⁴ cells/ml) in 24-well culture plates (Costar) and incubated at 37° C. under 5% CO₂.

For the binding assay the following standard procedure was followed. Upon reaching confluency, the cells were washed with the standard medium (Eagles medium without supplements), and 900 μl of the standard medium was added to the cells. Cells were inoculated with 100μl bacteria, 50 μl of strain Newman and 50 lμof AH12, to obtain a final concentration of 10⁷ bacteria per ml. After incubation for 2 hours at 37 C. and under 5% CO₂, the wells were washed 3 times with PBS. Two hundred μl of 10% trypsin was added to the wells to detach the cells, which were subsequently lysed by the addition of 800 μl of sterile water. The bacteria (both adherent and internalized) were serially diluted and plated on Blood agar plates. After 24 hours incubation, at least 200 colonies were picked from the Blood agar plates onto LB plates containing 4μg/ml of erythromycin and incubated for 24 hours at 37° C. to determine the ratio between the two strains (only AH12 is erythromycin resistant). The exact ratio between the two strains before adherence was determined in the same way.

For determination of internalization, lysostaphine at a final concentration of 20 μg/ml was added for 30 min to kill extracellular bacteria before the trypsin step. Thus only internalized bacteria are enumerated. The killing effect of lysostaphine was routinely checked in control wells at each experiment. The ratio between Newman and AH12 was determined by picking colonies onto erythromycin plates as above.

For clinical isolates (L12, L40, L167, U35, U61 and U98) adherence and internalization assays were performed in the same way. Strain Cowan 1 was included together with each strain and was given a relative value of 1 for adherence and internalization to which the clinical isolates were compared.

Adherence and internalization of S. aureus in the presence of Eap. Fibroblasts cells were cultured as in the adherence/internalization assay. Fifty μl of strain Newman O.N culture (10⁷ bacteria/ml) was pre-incubated for 30 min at 37° C. with 50 μl of Eap protein (80 μg/ml). The bacteria were then added to the cells in the wells. Control wells were inoculated with bacteria and PBS. After incubation for 2 hours at 37° C. and under 5% CO₂ the same procedure as for the adherence and internalization assay was performed. Bacteria were serially diluted and plated on Blood agar plates to determine viable counts.

IgG against Eap and GST-D. Sheep were immunized with Eap or GST-D. The latter is a fusion protein encompassing glutathion-S-Transferase and 3 binding domains from the fibronectin binding protein from S. aureus (15). Of each antigen, 150 μg in Freunds complete adjuvant was given intramuscularly. Booster doses were given two and four weeks later using Freunds incomplete adjuvant. Blood samples were taken two weeks after the last booster. A protein G Sepharose 4 Fast Flow (Pharmacia, Uppsala) was used to obtain IgG using the procedure recommended.

Adherence and Internalization in the presence of antibodies against Eap. Fibroblasts cells were cultured as in the adherence/internalization assay. Fifty μl of strain Newman O.N culture (10⁷ bacteria/ml) was pre-incubated for 30 min at 37° C. with 50 μl of Eap-Antibodies (8 mg/ml). Control wells were inoculated with bacteria and pre-serum (7 mg/ml). The bacteria were then added to the cells in the wells. After incubation for 2 hours at 37° C. and under 5% CO₂the same procedure as for the adherence and internalization assay was performed. Bacteria were serially diluted and plated on Blood agar plates to determine viable counts.

### RESULTS

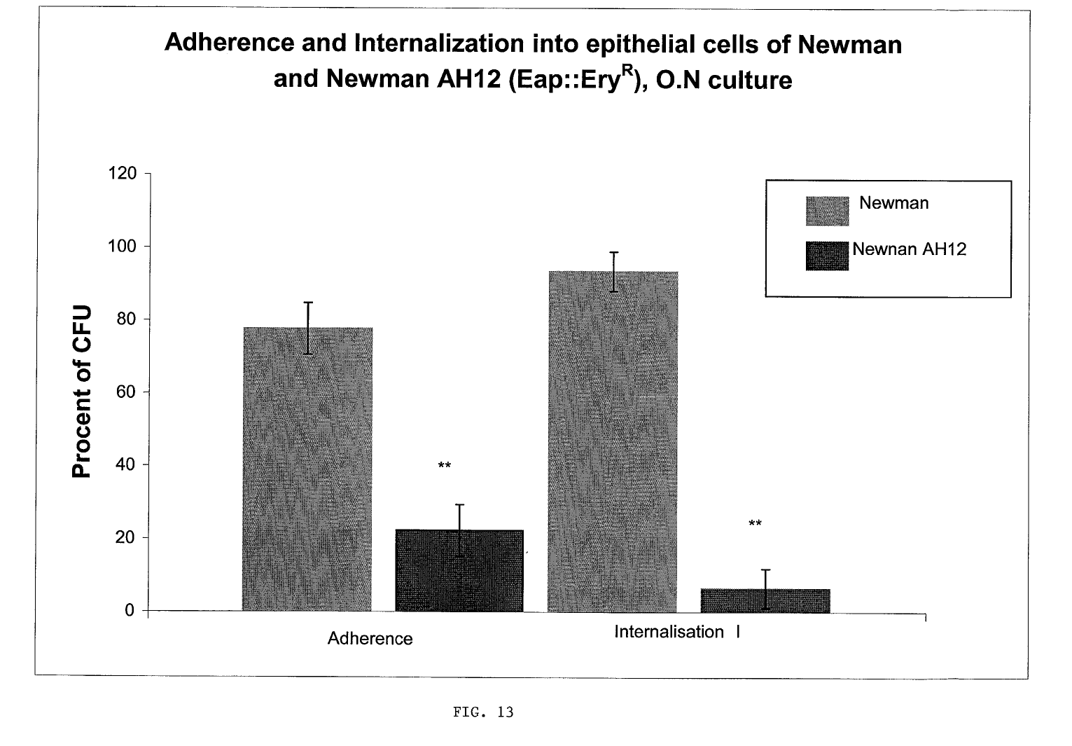

Adherence and internalization of S. aureus strain Newman and Newman AH12 to fibroblasts and epithelial cells: We have demonstrated that lack of the eap gene could reduce the adherence of S. aureus to fibroblasts as shown in FIG. 12. In the present experiment we address the question whether internalization of the eap-mutant was also reduced. A confluent layer of fibroblasts were inoculated with a mixture of S. aureus Newman/Newman AH12 (eap:: Ery^{R}) and incubated for 2 hours. Overnight cultures of Newman and AH12 were used since Eap is best expressed in a post-exponential phase and expression of FnBPs are low (14). Among the internalized bacteria strain Newman was clearly dominating over AH12 as shown in FIG. 12 (p<0.05).

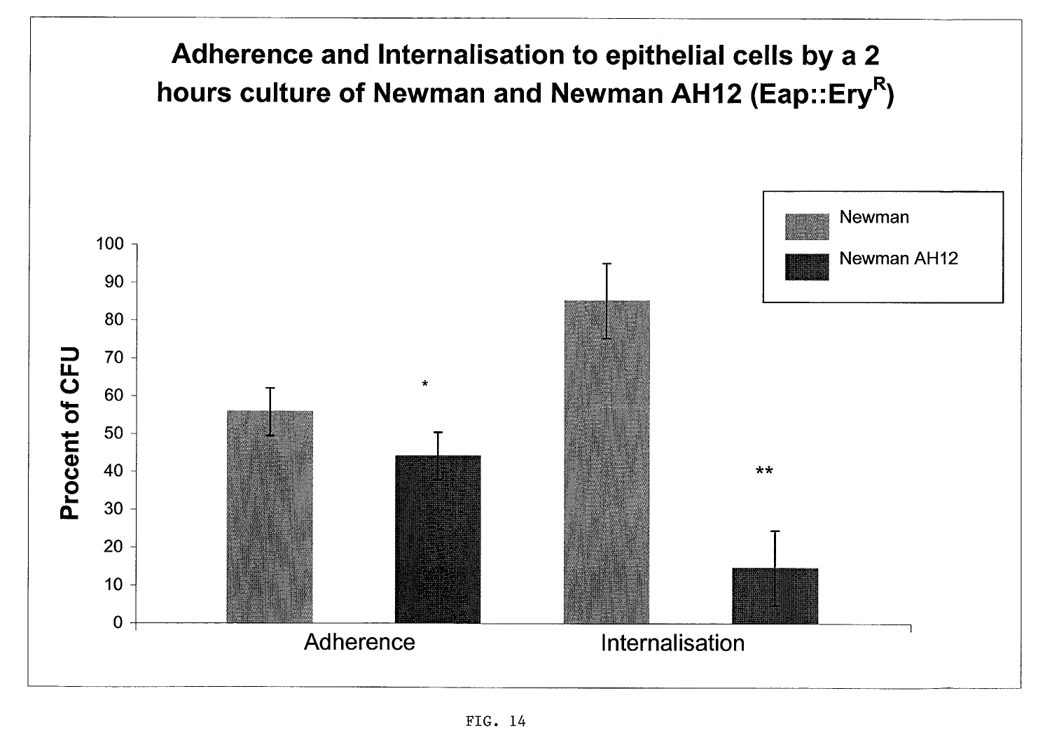

To exclude the possibility of cell specificity, also epithelial cells were subjected to the adherence and internalization assay. After incubation with the epithelial cells, a significant dominance of Newman over AH12 could be seen both in binding (p<0.05) and internalization (p<0.05) as shown in FIG. 13.

FnBPs have been shown to promote the internalization process of S. aureus into eukaryotic cells (10) (11) (12). FnBPs are cell-surface localized proteins that are best expressed in very early exponential phase. To allow sufficient expression of FnBPs, 2 hours cultures of strains Newman and AH12 were also used in adherence and internalization assays on epithelial cells. FIG. 14 shows that strain Newman is again dominating in both adherence (p<0.05) and internalization (p<0.05) over AH12, although to a lesser extent than what was the case with overnight cultures.

Adherence and internalization in the presence of externally added Eap. The above experiments showed that lack of Eap reduces adherence and internalization. Therefore, we wanted to test whether addition of external Eap could stimulate these events. Using the adherence and internalization assays on fibroblasts, bacteria were pre-treated with Eap prior to addition to the cells. Adherence of both strains Newman and AH12 is significantly enhanced (p<0.01) by addition of Eap, confirming our previous finding (5). Internalization of both strains is also significantly enhanced (p<0.01).

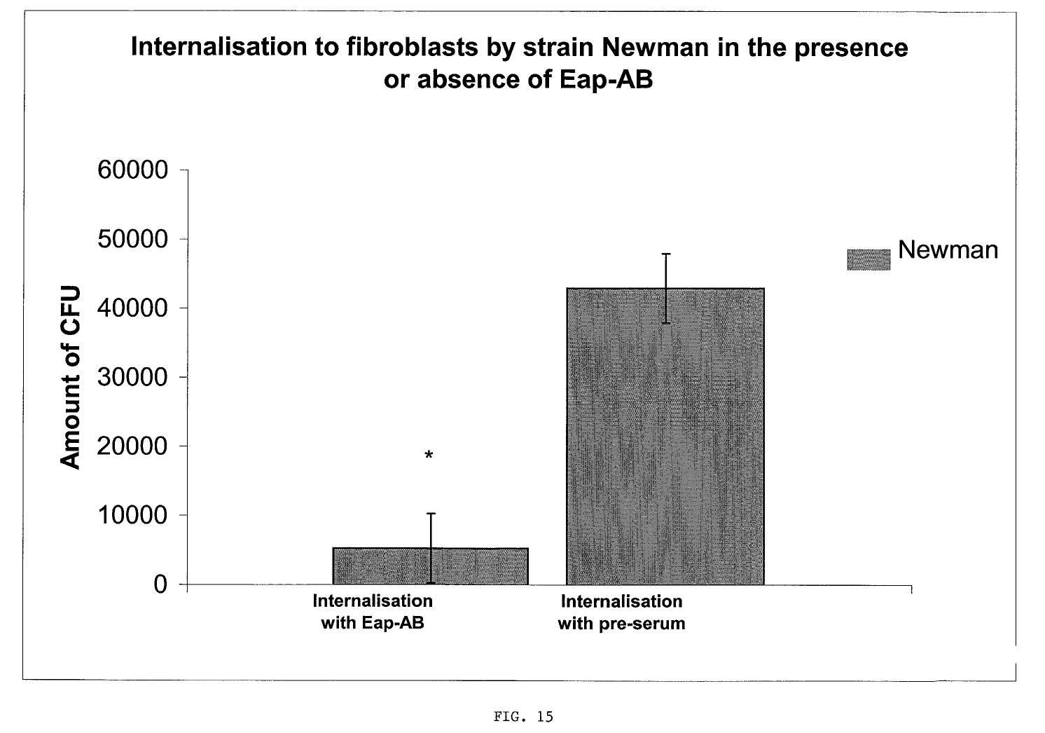

Reduced internalization of S. aureus in the presence of antibodies against Eap. Addition of external Eap could enhance the adherence and internalization both of strains Newman and AH12. In the next experiment the aim was to see if antibodies against Eap could block the internalization process. Using the internalization assay on fibroblasts or epithelial cells, strain Newman was pre-treated with antibodies against Eap prior to addition to the cells. FIG. 15 shows that these antibodies significantly reduce the internalization (p<0.05). Adherence of strain Newman to these cells is also reduced by addition of antibodies against Eap (data not shown).

It has been convincingly shown that fibronectin binding is a major factor promoting internalization of S. aureus into eukaryotic cells. We therefore tried the above experimental approach with antibodies against the D-domain on FnBP. Surprisingly, these antibodies were unable to block adherence or internalization of strain Newman into fibroblasts (data not shown).

Adherence and internalization of strain from clinical isolates. Six S.aureus clinical isolates were tested for adherence and internalization into fibroblasts to assess the variation. For each strain tested, Cowan 1 was included and was given the relative value of 1 for both adherence and internalization. As Table 1 shows, there was a big variation between the clinical isolates in adherence and internalization into fibroblasts. There was obviously no correlation between adherence and internalization. Although adherence is needed for internalization to occur, the lack of correlation shows that it is not sufficient for internalization.

TABLE 1
Adherence and Internalization of six S. aureus
clinical isolates into fibroblasts

| Strains | Adherence | Internalization |
|---|---|---|
| Cowan 1 | 1 | 1 |
| Newman | 0.63 | 0.15 |
| L12 | 2.07 | 0.14 |
| L40 | 1.24 | 0.38 |
| L167 | 0.74 | 1.04 |
| U35 | 2.89 | 1.83 |
| U61 | 1.19 | 0.46 |
| U98 | 0.45 | 0.37 |

Strain Cowan 1 was given a relative value of 1 for adherence and internalization. The ratio for Newman and the six clinical isolates is estimates as the average of adherence or internalization of each clinical isolate divided by the average of adherence or internalization of Cowan 1. Each experiment was performed twice.

### References

1. McDevitt, D., P. Francois, P. Vaudaux, and T. J. Foster. 1994. Molecular characterization of the clumping factor (fibrinogen receptor) of Staphylococcus aureus. Mol Microbiol. 11 (2):237-48.

2. Ni Eidhin, D., S. Perkins, P. Francois, P. Vaudaux, M. Hook, and T. J. Foster. 1998. Clumping factor B (ClfB), a new surface-located fibrinogen-binding adhesin of Staphylococcus aureus. Mol Microbiol. 30(2):245-57.

3. Phonimdaeng, P., M. O'Reilly, P. Nowlan, A. J. Bramley, and T. J. Foster. 1990. The coagulase of Staphylococcus aureus 8325-4. Sequence analysis and virulence of site-specific coagulase-deficient mutants. Mol. Microbiol. 4(3):393-404.

4. Palma, M., D. Wade, M. Flock, and J.-l. Flock. 1998. Multiple binding sites in the interaction between fibrinogen and an extracellular fibrinogen binding protein from Staphylococcus aureus. J. Biol. Chem.:Submitted.

5. Palma, M., A. Haggar, and J.-l. Flock. 1999. Adherence of Staphylococcus aureus is enhanced by an endogenous secreted protein with broad binding activity. J. Bacteriol. 181:2840-2845.

6. Bayles, K. W., C. A. Wesson, L. E. Liou, L. K. Fox, G. A. Bohach, and W. R. Trumble. 1998. Intracellular Staphylococcus aureus escapes the endosome and induces apoptosis in epithelial cells. Infect Immun. 66(1):336-42.

7. Menzies, B. E., and I. Kourteva. 1998. Internalization of Staphylococcus aureus by endothelial cells induces apoptosis. Infect Immun. 66(12):5994-8.

8. Wesson, C. A., L. E. Liou, K. M. Todd, G. A. Bohach, W. R. Trumble, and K. W. Bayles. 1998. Staphylococcus aureus Agr and Sar global regulators influence internalization and induction of apoptosis. Infect Immun. 66(11):5238-43.

9. Jevon, M., C. Guo, B. Ma, N. Mordan, S. P. Nair, M. Harris, B. Henderson, G. Bentley, and S. Meghji. 1999. Mechanisms of internalization of Staphylococcus aureus by cultured human osteoblasts. Infect Immun. 67(5):2677-81.

10. Dziewanowska, K., J. M. Patti, C. F. Deobald, K. W. Bayles, W. R. Trumble, and G. A. Bohach. 1999. Fibronectin binding protein and host cell tyrosine kinase are required for internalization of Staphylococcus aureus by epithelial cells. Infect Immun. 67(9):4673-8.

11. Sinha, B., P. P. Francois, O. Nusse, M. Foti, O. M. Hartford, P. Vaudaux, T. J. Foster, D. P. Lew, M. Herrmann, and K. H. Krause. 1999. Fibronectin-binding protein acts as Staphylococcus aureus invasin via fibronectin bridging to integrin alpha5beta1. Cell Microbiol. 1(2):101-17.

12. Peacock, S. J., T. J. Foster, B. J. Cameron, and A. R. Berendt. 1999. Bacterial fibronectin-binding proteins and endothelial cell surface fibronectin mediate adherence of Staphylococcus aureus to resting human endothelial cells. Microbiology. 145(Pt 12):3477-86.

13. Bodén, M., and J.-l. Flock. 1989. Fibrinogen binding protein/Clumping factor from Staphylococcus aureus. Inf. Imm. 57:2358-2363.

14. Bodén, M., and J.-l. Flock. 1992. Evidence for three different fibrinogen-binding proteins with unique properties from Staphylococcus aureus strain Newman. Microbial Pathogenesis. 12:289-298.

15. Brennan, F. R., T. D. Jones, M. Longstaff, S. Chapman, T. Bellaby, H. Smith, F. Xu, W. D. Hamilton, and J. I. Flock. 1999. Immunogenicity of peptides derived from a fibronectin-binding protein of S. aureus expressed on two different plant viruses. Vaccine. 17(15-16):1846-57.

1. A new fibrinogen binding protein derived from Staphylococci having a molecular weight of 60 kDa.

2. Hybrid-DNA-molecule comprising a nucleotide sequence from S. aureus coding for a protein or polypeptide having fibrinogen binding activity.

3. Plasmid or phage comprising a nucleotide sequence from S. aureus coding for a protein or polypeptide having fibrinogen binding activity.

4. An E. coli strain expressing said fibrinogen binding protein.

5. A microorganism transformed by recombinant DNA molecule of claim 2.

6. Hybrid-DNA-molecule according to claim 2, comprising the following nucleotide sequence:
GAGCGAAGGA TACGGTCCAA GAGAAAAGAA ACCAGTGAGT
ATTAATCACA ATATCGTAGA GTACAATGAT GGTACTTTTA
AATATCAATC TAGACCAAAA TTTAACTCAA CACCTAAATA
TATTAAATTC AAACATGACT ATAATATTTT AGAATTTAAC
GATGGTACAT TCGAATATGG TGCACGTCCA CAATTTAATA
AACCAGCAGC GAAAACTGAT GCAACTATTA AAAAAGAACA
AAAATTGATT CAAGCTCAAA ATCTTGTGAG AGAATTTGAA
AAAACACATA CTGTCAGTGC ACACAGAAAA GCACAAAAGG
CAGTCAACTT AGTTTCGTTT GAATACAAAG TGAACAAAAT
GGTCTTACAA GAGCGAATTG ATAATGTATT AAAACAAGGA
TTAGTGAGA

7. A method for producing a fibrinogen binding protein or polypeptide wherein a) at least one hybrid-DNA molecule according to claim 2, is introduced into a microorganism, b) said microorganism is cultivated in a growth promoting medium, and c) the protein thus formed is isolated.

8. A fibrinogen binding protein or polypeptide comprising at least one amino acid sequence
SEGYGPREKK PVSINHNIVE YNDGTFKYQS RPKFNSTPKY
IKFKHDYNIL EFNDGTFEYG ARPQFNKPAA KTDATIKKEQ
KLIQAQNLVR EFEKTHTVSA HRKAQKAVNL VSFEYKVKKM
VLQERIDNVL KQGLVR

9. Pharmaceutical composition for the inhibition of Staphylococci binding to fibrinogen comprising a fibrinogen binding protein of claim 1 in combination with a pharmaceutically acceptable carrier.

10. Method for inhibition of Staphylococci binding to fibrinogen in mammals including humans, by administering a therapeutically and/or prophylactically effective amount of a fibrinogen binding protein of claim 1 to a mammal in need of such treatment.

11. Method for passive immunization against Staphylococcal infection, comprising administering to a mammal antibodies against a fibrinogen binding protein of claim 1 in an amount sufficient to provide passive immunization.


SEQUENCE LISTING (15 sequences)

SEQ ID NO: 1 - protein, 20 aa
IVTKDYSKESRVNENSKYGT

SEQ ID NO: 2 - protein, 20 aa
IVTKDYSKESRVNEKSKKGA

SEQ ID NO: 3 - protein, 20 aa
IVTKDYSGKSQVNAGSKNGT

SEQ ID NO: 4 - protein, 20 aa
IVTKDYSGKSQVNAGSKNGT

SEQ ID NO: 5 - protein, 20 aa
SEGYGPREKKPVSINHNIVE

SEQ ID NO: 6 - protein, 8 aa
MYPEKKPV

SEQ ID NO: 7 - DNA (genomic), 408 bp; single-stranded, linear
gagcgaaggatacggtccaagagaaaagaaaccagtgagtattaatcacaatatcgtagagtacaatgatggtacttttaaatatcaatctagaccaaaatttaactcaacacctaaatatattaaattcaaacatgactataatattttagaatttaacgatggtacattcgaatatggtgcacgtccacaatttaataaaccagcagcgaaaactgatgcaactattaaaaaagaacaaaaattgattcaagctcaaaatcttgtgagagaatttgaaaaaacacatactgtcagtgcacacagaaaagcacaaaaggcagtcaacttagtttcgtttgaatacaaagtgaagaaaatggtcttacaagagcgaattgataatgtattaaaacaaggattagtgag

SEQ ID NO: 8 - protein, 136 aa
SEGYGPREKKPVSINHNIVEYNDGTFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVR

SEQ ID NO: 9 - DNA (genomic), 1009 bp; single-stranded, linear
  CDS 157..654
  CDS 804..1007
gactagtgtataagtgctgatgagtcacaagatagataactatattttgtctatattataaagtgtttatagttaattaataattagttaatttcaaaagttgtataaataggataacttaataaatgtaagataataatttggaggataattaacatgaaaaataaattgatagcaaaatctttattaacaatagcggcaattggtattactacaactacaattgcgtcaacagcagatgcgagcgaaggatacggtccaagagaaaagaaaccagtgagtattaatcacaatatcgtagagtacaatgatggtacttttaaatatcaatctagaccaaaatttaactcaacacctaaatatattaaattcaaacatgactataatattttagaatttaacgatggtacattcgaatatggtgcacgtccacaatttaataaaccagcagcgaaaactgatgcaactattaaaaaagaacaaaaattgattcaagctcaaaatcttgtgagagaatttgaaaaaacacatactgtcagtgcacacagaaaagcacaaaaggcagtcaacttagtttcgtttgaatacaaagtgaagaaaatggtcttacaagagcgaattgataatgtattaaaacaaggattagtgagataatacttctgtcattattttaagttcaaaataatttaatattatattattttttattaataaaacgactatgctatttaatgccaggttaatgtaactttcctaaaattgactatataatcgttaagtatcaattttaaggagagtttacaatgaaatttaaaaaatatatattaacaggaacattagcattacttttatcatcaactgggatagcaactatagaagggaataaagcagatgcaagtagtctggacaaatatttaactgaaagtcagtttcatgataaacgcatagcagaagaattaagaactttacttaacaaatcgaatgtatatgcattagctgcaggaagctt

SEQ ID NO: 10 - DNA (genomic), 781 bp; single-stranded, linear
atagataactatattttgtctatattataaagtgtttatagttaattaataattagttaatttcaaaagttgtataaataggataacttaataaatgtaagataataatttggaggataattaacatgaaaaataaattgatagcaaaatctttattaacaatagcggcaattggtattactacaactacaattgcgtcaacagcagatgcgagcgaaggatacggtccaagagaaaagaaaccagtgagtattaatcacaatatcgtagagtacaatgatggtacttttaaatatcaatctagaccaaaatttaactcaacacctaaatatattaaattcaaacatgactataatattttagaatttaacgatggtacattcgaatatggtgcacgtccacaatttaataaaccagcagcgaaaactgatgcaactattaaaaaagaacaaaaattgattcaagctcaaaatcttgtgagagaatttgaaaaaacacatactgtcagtgcacacagaaaagcacaaaaggcagtcaacttagtttcgtttgaatacaaagtgaagaaaatggtcttacaagagcgaattgataatgtattaaaacaaggattagtgagataatacttctgtcattattttaagttcaaaataatttaatattatattattttttattaataaaacgactatgctatttaatgccaggttaatgtaactttcctaaaattgactatataatcgttaagtatcaattttaaggagagtttacaatgaaattt

SEQ ID NO: 11 - DNA (genomic), 785 bp; single-stranded, linear
atagatagctatattcagtctatattataaagtgtttatagttaattaataattagttaatttcaaaagttgtataaataggataacttaataaatgtaagataataatttggaggataattgacatgaaaaatgcattgatagcaaaatctttattaacattagcggcaataggtattactacaactacaattgcgtcaacagcagatgcgagcgaaggatacggtccaagagaaaagaaaccagtgagtattaatcacaatatcgtagagtacaatgatggtacttttaaatatcaatctagaccaaaatttaactcaacacctaaatatattaaattcaaacatgactataatattttagaatttaacgatggtacattcgaatatggtgcacgtccacaatttaataaaccagcagcgaaaactgatgcaactattaaaaaagaacaaaaattgattcaagctcaaaatcttgtgagagaatttgaaaaaacacatactgtcagtgcacacagaaaagcacaaaaggcagtcaacttagtttcgtttgaatacaaagtgaagaaaatggtcttacaagagcgaattgataatgtattaaaacaaggattagttaaataaaacttcaatcgttgctgttatctggaaataattaattaaatgttatgttaatttttgttaatgaaaaaagtaatctatttaatgacaggttaatgtaattgtcctgaaattgactatatactcagtaagtatcaattttaaggagagcttataatgaaattt

SEQ ID NO: 12 - protein, 165 aa
MKNKLIAKSLLTIAAIGITTTTIASTADASEGYGPREKKPVSINHNIVEYNDGTFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVR

SEQ ID NO: 13 - protein, 165 aa
MKNALIAKSLLTLAAIGITTTTIASTADASEGYGPREKKPVSINHNIVEYNDGTFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVK

SEQ ID NO: 14 - protein, 136 aa
SEGYGPREKKPVSINHNIVEYNDGSFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVR

SEQ ID NO: 15 - protein, 177 aa
ASQYGPRPQFNKTPKYVKYRDAGTGIREYNDGTFGYEARPRFNKPSETNAYNVTTHANGQVSYGARPTYKKPSETNAYNVTTHANGQVSYGARPTQNKPSETNAYNVTTHGNGQVSYGARQAQNKPSKTNAYNVTTHANGQVSYGARPTYKKPSKTNAYNVTTHADGTATYGPRVTK